(19) United States
(12) Patent Application Publication
(10) Pub. No.: US 2014/0345394 A1
(43) Pub. Date: Nov. 27, 2014

(54) PORTABLE SOIL TESTING APPARATUS AND METHOD

(71) Applicant: Rhett Warren Schildroth, North Liberty, IA (US)

(72) Inventor: Rhett Warren Schildroth, North Liberty, IA (US)

(21) Appl. No.: 14/253,926

(22) Filed: Apr. 16, 2014

Related U.S. Application Data
(60) Provisional application No. 61/827,620, filed on May 26, 2013.

(51) Int. Cl. G01N 33/24

(52) U.S. Cl. CPC G01N 33/24; G01N 2033/245
USPC 73/863

(57) ABSTRACT

A portable soil testing apparatus and method including a device to mix a soil sample in a cup with water to create a slurry, immersing a sensor in the slurry to detect a soil property and recording the soil property along with location information. The soil testing apparatus includes a frame, a computer secured within the frame, the computer operatively connected to a touch screen display and a pump. The pump providing water from a tank and into a cup containing a soil sample. A stirring station including a stirring rod and a motor is controlled by the computer and mixes the soil sample and water to create a slurry. A sensor is then immersed in the slurry to detect a desired soil property. The soil property data is then recorded by the computer on a storage medium, transmitted to a user or displayed.

### CROSS-REFERENCE TO RELATED APPLICATIONS

This application claims priority under 35 U.S.C. §119 to provisional application Ser. No. 61/827,620 filed on May 26, 2013, herein incorporated by reference in its entirety.

### FIELD OF THE INVENTION

The present invention relates generally to an apparatus method of testing and analyzing soil. In particular, the present invention relates to an apparatus and method for testing soil on location, preferably by an easily transportable self-contained unit. The unit may be taken to the soil location by the user who collects the soil sample for testing. Alternatively, the unit may be incorporated into an autonomous vehicle that can take soil samples at desired locations as instructed.

### BACKGROUND OF THE INVENTION

Location based soil testing and analysis allows agricultural growers and providers to tailor seed variety and chemical applications to the appropriate growing condition. Here to for, it has been difficult to obtain soil properties without the use of either large soil collection machines or external laboratories. The use of external laboratories for soil testing is particularly problematic. For example, when external labs are used, a soil sample must be sufficiently large enough to enable proper testing, the soil sample must be sent away from the site, meaning it must be appropriately labeled and tracked so that the later obtained test results can accurately be tied to the location for which the soil samples were taken, and the soil is unlikely to be returned to the same location, if at all. Additionally, soil testing at external labs is typically done by drying out the soil sample, which can skew results due to changes in the soil condition that may occur during drying. Changes may also occur to the soil sample during the shipping process.

Automated soil collection devices have also been developed that analyze soil one-the-go. Such automated soil analyzing machines are typically large attachments that must be pulled by the grower through the field of interest. Not only are such machines extremely expensive, but they take time to set up, attach to the tractor or other vehicle and can generally only be used when crops are not present. This minimizes their uses to pre-planting or post-planting timeframes, making it difficult to determine actual growing conditions throughout the plant's life cycle.

Further, previous attempts to provide on-the-go soil analysis and sampling typically test the soil in a solid state. This leads to potential damage to the analyzing sensors from rocks and other field debris. Prior inventions have attempted to address this issue. For example, the U.S. Pat. No. 7,216,555 attempts to address the potential for abrasion and damage to the sensors by providing pressure sensitive measurements—essentially allowing the sensors to move in response to rocks and other field debris. While this may help to minimize the damage, the additional shock absorbing sensor mounts add expense, weight and complexity to the device. Moreover, because the device measures the soil in solid form and rocks and field debris can vary the position of the sensors, is questionable whether the test results accurately reflect the true composition of the soil that is sampled. There's therefore a need for a soil testing apparatus and method which avoids the expense, complexity, and potential inaccuracies of apparatus discussed in the '555 patent.

Others have attempted to provide robotic vehicles with soil testing labs built in. For example, U.S. Patent Application Publication Number 2003/0112152 discloses a robotic vehicle and method for soil testing. This system is a dedicated autonomous system. While such systems can determine soil properties by location autonomously, they are not easily transportable and are expensive. Moreover, a user of such systems cannot easily randomly test soil properties on the fly and they cannot retest soil conditions if a rogue sample yields odd results. Additionally, farmers may want to know the soil condition of a particular area of interest while they are physically present in the field. If the farmer were using the autonomous system of '152 application, the farmer would then have to call out the robot, direct it to the specific location, wait for the robot to arrive, perform testing and upload results so that the farmer could understand the soil conditions at the farmer's present location. It is desirable to provide the farmer with the ability to test soil conditions on the go without the need to call up a dedicated soil testing autonomous system.

It is also desirable to have a system that is compact enough for easy transport to and within a field, tolerates dust and temperature fluctuations, can absorb shock loads and vibrations and allows for the sensor to be operated in the field at any time. It is therefore desirable to have a soil testing apparatus and method that addresses these other problems in the art.

Thus, there is a need in the art for an apparatus and method for soil testing that is portable, easy to use, prevents damage to sensors and allows for testing at a farmer's desired location.

### SUMMARY OF THE INVENTION

Thus, it is a principal object, feature, and/or advantage of the present inventions to provide an apparatus or method that overcomes the deficiencies in the art.

It is therefore an object, feature and/or benefit for the present invention to provide an apparatus and method for soil testing that allows for soil testing at a desired location. It is another object, feature and/or benefit of the present invention to provide an apparatus and method for soil method for soil testing that can do soil testing on site and coordinate soil test results with location data.

Still another object, feature, and/or advantage of the present invention to provide an apparatus and method for soil testing that is less expensive than other alternatives.

It is a further object, feature, and/or advantage of the present invention to provide an apparatus and method for soil testing that is easily used by the farmer.

These and/or other objects, features, and advantages of the present invention will be apparent to those skilled in the art. The present invention is not to be limited to or by these objects, features, and advantages. No single embodiment need provide each and every object, feature, and/or advantage.

An aspect of the present invention allows for a soil testing apparatus to be taken to a desired location and used by a farmer to perform a soil analysis on-site. The preferred apparatus includes a soil sample cup into which a soil sample is placed. This soil sample is then mixed with water and stirred to create a slurry. Preferably, the soil testing apparatus includes a mixing station that is separate and apart from the soil sensor station. However, the soil mixing station and the soil sensor station may be combined to perform both mixing and sensing at the same location. At the soil mixing station, water can be mixed with the soil sample and a stirrer is present such that the user can place the cup into the soil mixing station, operate the stirrer and create the soil slurry for testing. Then, the user moves the soil sample slurry to the soil testing station.

At the soil testing station, one or more soil sample sensors are present. The soil sample sensors can detect certain characteristics of the soil sample slurry. The user simply places the cup containing the soil sample slurry into the soil sampling station where the sensor is present. The soil sensor is then operated to detect the properties of interest and then the soil sample cup can be removed and the soil sample discarded or placed back into the field.

Data from the soil sampling station and soil sensor is then routed into the data acquisition system. The data acquisition system includes a processor that collects data from the sensor and soil sampling station. Data is also collected from location sensors to coordinate soil sampling data with a specific location. This data is then stored into the memory of the system and may be output to an on-board printer, saved for later download, or routed via an antenna for wireless transmission to a base station or portable device, such as farmer's cell phone. The system may be powered by an onboard battery or maybe powered by the user's vehicle via a power convertor. Additionally, data from the soil testing may be displayed on an on-board display. This provides near real time feedback for the farmer at the location of the soil test. In another embodiment, the soil testing apparatus of the present invention can be secured to a tractor, combine, sprayer, or other vehicle for the farmer to use as desired and may be incorporated into an autonomous system.

Among soil properties of interest are soil pH, potassium content, phosphorous content, cation exchange capacity (CEC) and organic carbon. However, perhaps of primary interest is the analysis of the nitrogen content of the soil. Accordingly, our sensing system is designed particularly with the analysis of nitrogen in mind, although it might readily be adapted to the analysis of other soil properties such as soil pH, potassium content, phosphorous content, cation exchange capacity, organic carbon content, etc. without departing from the invention. Among problems to be addressed in the design of the system were such matters as selection of design alternatives of the sample presentation mechanism, the mechanism to mix the soil and water into a soil slurry, the design of the sensor and data acquisition systems, the processing and analysis of the data acquired, the means to record the data, the means for the operator to interact with the system, the design such that it is compact enough for easy transport to and within a field, and tolerance of dust, temperature fluctuations, shock loads, and vibration, such as would be encountered if the sensor were operated in the field.

### BRIEF DESCRIPTION OF THE DRAWINGS

FIG. 1 is a front view of one embodiment of the apparatus for soil testing of the present invention.

FIG. 2 is a side view of the embodiment of FIG. 1 of the apparatus for soil testing of the present invention as secured to a portable base.

FIG. 3 is a rear view of the soil testing apparatus of FIG. 1 of the present invention.

Various embodiments of the present invention will be described in detail with reference to these drawings, where in like referencing will represent like parts throughout the several views. Reference to various embodiments does not limit the scope of the invention. Figures represented here and are not limitations to the various embodiments according to the invention and are presented for exemplary illustration of the invention only.

### DETAILED DESCRIPTION OF THE PREFERRED EMBODIMENTS

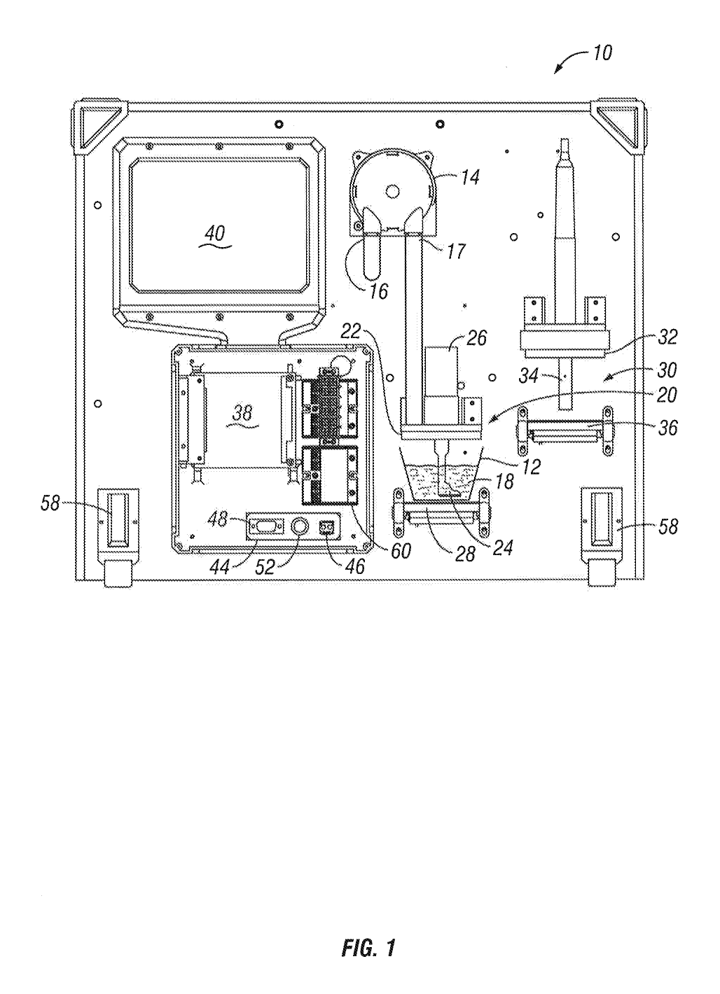

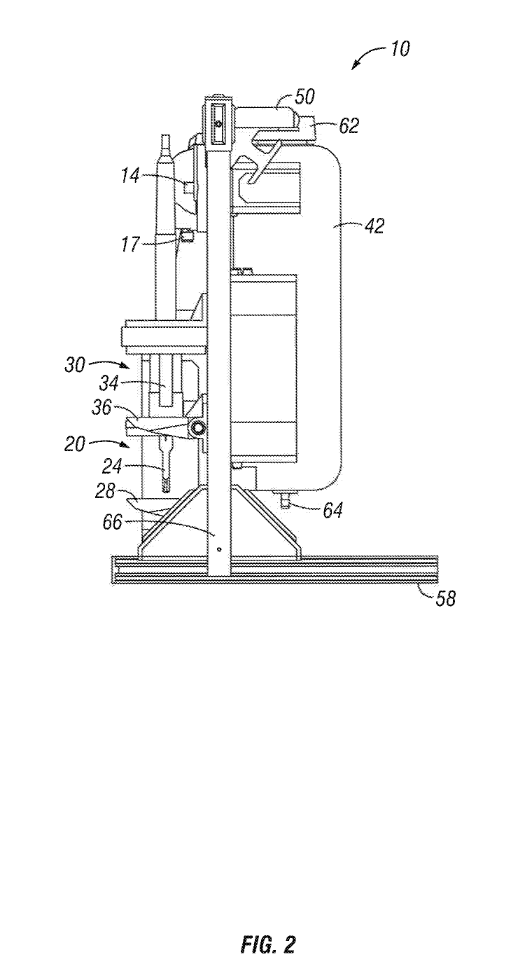

Now referring to FIGS. 1-3, the reference 10 generally designates the apparatus for soil testing of the present invention. Initially, a soil sample is gathered and put into a cup 12, such as a disposable Dixie brand cup. Using a standardized disposable cup, such as the well-known 5 oz. Dixie brand cups, keeps the sample size to a minimum while also keeping the soil sample container at a low cost. The apparatus 10 preferably includes a pump 14, such as a peristaltic pump with an input 16 connected to the tank 42 by a hose and an outlet 17 that adds preferably deionized water into the cup 12 at the stirring station 20. A calibrated amount, such as 2 oz., can be added to ensure a general consistency of the soil sample slurry 18. The calibration can be adjusted by controlling the duration the pump 14 operates.

The cup 12 with the soil and water in it is placed into the soil stirring station 20. The soil stirring station 20 generally includes a cup support platform 28, a stirring rod 24, and a preferably reversible motor 26 operatively connected to turn the stirring rod 24. The cup support platform 28 is preferably mounted on a spring loaded hinge which allows the user to press the cup support platform 28 down, insert the cup 12 and allow the spring loaded hinge to raise the cup support platform 28 back up to contact and support the cup 12. Alternatively, the cup support platform 28 may slide up and down, either manually or operationally using a slide track and motor (not shown) and a cup holding ring 22 may be included. Once the water has been added by the pump 14 at the soil stirring station 20, the motor 26 turns the stirring rod 24 to mix the soil and water to create the soil sample slurry 18. The stirring rod 24 is preferably made from a resilient plastic or metal and is preferably easily replaceable.

By having the stirring rod 24 mix the soil and water, rocks and field debris can settle out of the slurry, thus minimizing their potential impact on more sensitive components, such as the sensor 34. Thus, it is preferred that the soil stirring station 20 be separate and apart from the soil sensor station 30. Alternatively, if a more compact design is desirable, the soil stirring station 20 and soil sensor station 30 can be combined where both the stirring rod 24 and the sensor 34 will fit into the cup 12 at a single location. However, this increases the chance of damaging the sensor 34 with rocks or field debris commonly found in soil. To avoid such damage, the sensor 34 may be removeably inserted into the cup 12 when testing is desired and removed from the cup 12 when not in use, such as during the stirring operation. Alternatively still, multiple sensors can be used with one sample. Either multiple sensors can be immersed in the slurry at the same time or, each sensor 34 may be inserted and removed automatically by a motor and track assembly (not shown) controlled by the computer 38 to position and operate the appropriate sensor 34 at the appropriate time.

At the soil sensor station 30, the cup 12 is placed onto the cup support platform 36 and the sensor 34 is sufficiently immersed into the soil sample slurry 18 for the sensor 34 to operate effectively. The cup support platform 36 is preferably mounted on a spring loaded hinge which allows the user to press the cup support platform 36 down, insert the cup 12 and allow the spring loaded hinge to raise the cup support platform 36 back up to contact and support the cup 12. Alternatively, the soil sensor station 30 may include a cup support platform 36, which may slide up and down, either manually or operationally using a slide track and motor (not shown) and a cup holding ring 32 may be included.

The sensor 34 may be any known sensor capable of reading a desired soil characteristic or measurement. For example, the sensor 34 can be an ion selective electrode, a near-infrared sensor to measure transmission or reflection of light in the near-infrared band, laser-induced-breakdown spectroscopy, or other light based spectroscopy as is discussed in U.S. Patent Application Publication No. 20130258317, which is incorporated herein by reference in its entirety. The sensor 34 is preferably easily interchangeable by providing an array of sensors with a common connector, such that the user can easily switch the sensors 34 to measure the property of interest in the soil slurry sample 18.

Preferably, the sensor 24 is operatively connected to an on-board computer 38. The on-board computer 38 is preprogrammed to account for the variety of sensors 34 available, to accept inputs as to the sensor 34 currently being used, and to perform necessary calculations based on the detections made by the sensor 34. If a single sensor 34 can be used to measure multiple soil properties, the computer 38 can adjust the calculations as needed to measure the property of interest. In this manner the soil sensor station is capable of measuring a variety of soil properties, such as analyzing the soil for different nutrients, including, but not limited to, potassium, phosphorus, nitrate nitrogen, ammonium nitrogen, manganese, bromide, Fluoride, Zinc, boron, molybdenum, salinity, pH, Humus (organic matter), calcium, magnesium, ammonia nitrogen, manganese, aluminum, nitrite nitrogen, sulfur, chloride, ferric iron, and copper.

The computer 38 preferably accepts inputs from and provides outputs to a touch screen display 40 to which it is operatively connected. While the display 40 is preferably a touch screen type of display, a non-touch screen display can be used in combination with other input means, such as buttons, a keyboard, or a remote input source, such as a user's smartphone. The computer may have a wireless transceiver operatively connected to it to enable communication over a cellular network, a wireless internet, a Bluetooth connection, or another type of wireless communication system through which a user's personal communication device may operate.

At the touch screen display 40, instructions can be displayed to walk the user through each step and track the user's progress. For example, when the user has a sample ready for the addition of water, the user can select the “add water” button. When the user then places the cup 12 in the stirring station 20, the user can select the “mix” button. After mixing, the user can transfer the cup 12 to the soil sensor station 30 for analysis and hit the “analyze” button.

If desired, the user can input the type of sensor 34 being used or simply request the measurement to be taken. For example, if the user asks for the nitrogen content of the sample 18, the computer 38 is programmed to know the sensor 34 being used will detect the amount of nitrogen in the sample 18. The computer 38 controls the system 10, responds to the user's inputs, displays the data, and saves the data preferably on a solid state data storage medium. Other types of data storage media may be used including standard hard drives and removable storage media, such as flash or USB drives. The computer 38 is preferably connected to a connector panel 44 that provides a power input 46, a GPS display input 48 and a GPS signal input 52, if desired. Power, may be provided to the power input 46 by a user's vehicle such as through a 110 volt connection or, alternatively, an on-board battery, such as a 12 volt battery, can be provided and integrated with the apparatus 10. If an on-board rechargeable battery is provided, the power input 46 can also recharge the battery as desired.

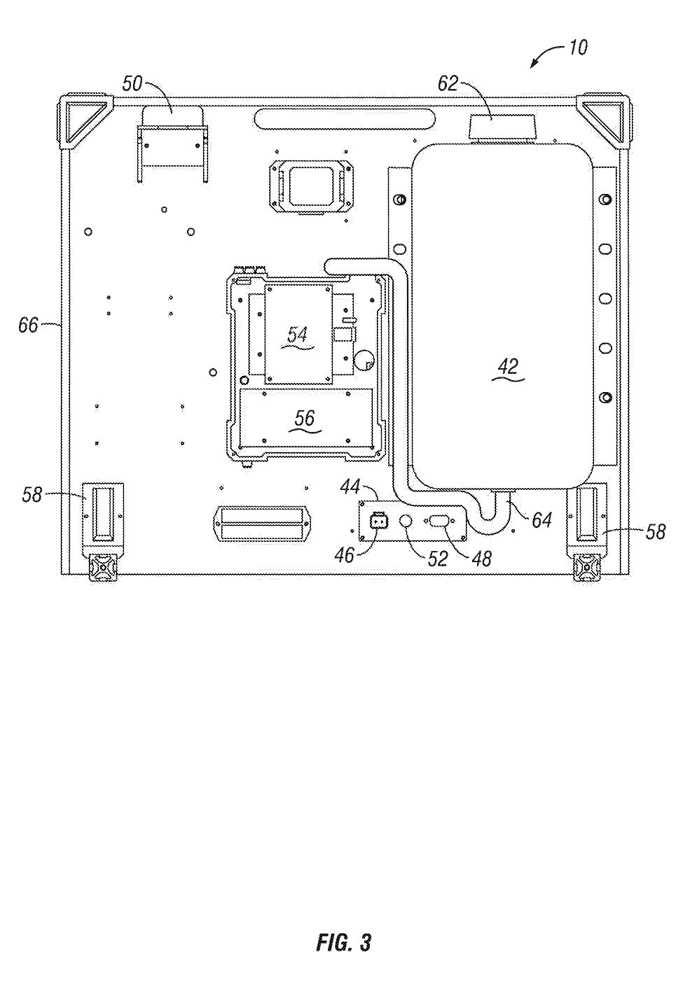

As shown in FIG. 3, a GPS antenna is also included and is operatively linked to the computer 38. This allows the computer 38 to record the location where the soil sample was analyzed. As the soil testing apparatus 10 is meant to be compact and portable, it is therefore presumed that the analysis takes place in close proximity to where the soil sample is taken and thus provides the user with data for the soil conditions at that location. A circuit board 54 can convert analog sensor information to a digital format and provides signal outputs to the motor controller board 56. The motor controller board 56 controls the voltage outputs to operate the pump 14, mixing motor 26 and any slide motors that may be present.

The water tank 42 is also shown in FIG. 3. The water tank 42 is designed to hold a sufficient supply of water for the amount of intended testing and may be refilled on the go. A cap 62 is provided to allow the water tank 42 to be refilled as necessary. Alternatively, the water tank 42 may be connected to a water source to ensure a constant supply of water, if, for example, the apparatus 10 is mounted onto a user's vehicle. A hose connection 64 is also preferably provided to connect the tank 42 to the pump 14. When no longer needed, the user can simply unhook the hose and allow the tank 42 to drain. This helps to minimize the weight of the apparatus 10 and thus makes it easier to transport.

Preferably, the apparatus 10 is secured within a frame 66 that is a rigid metal or plastic. The various components of the apparatus 10, such as the computer 38, the soil stirring station 20, the soil sensor station 30 and the water tank 42 are preferably secured within the frame 66 to a common support structure, such as a plastic board. The frame 66 preferably provides protection and may include rubber or soft plastic edges or corners to minimize impact shock to the apparatus 10. One or more legs 58 may also be included to allow the user to set the apparatus on a surface without fear of it falling over. Alternatively, a case may be included that surrounds the components of the apparatus 10 and provides protection from adverse weather, dust, and travel conditions.

As discussed above, the apparatus 10 of the present invention may be mounted to a tractor or other vehicle, if desired. Alternatively, the apparatus 10 of the present invention may be mounted to an autonomous vehicle that includes a soil sampling apparatus. For example, a mobile autonomous device can be programmed to go to a designated spot or series of spots, take soil samples from the field, and automatically analyze the soil for different nutrients, including, but not limited to, potassium, phosphorus, nitrate nitrogen, ammonium nitrogen, manganese, bromide, fluoride, Zinc, boron, molybdenum, salinity, pH, Humus (organic matter), calcium, magnesium, ammonia nitrogen, manganese, aluminum, nitrite nitrogen, sulfur, chloride, ferric iron, and copper by using the appropriate sensor 34, such as, for example, by measuring reflected or transmitted light in the near infrared, mid infrared, or visible light bands for the light corresponding to the desired nutrient; by using an ion selective electrode is inserted into to measure the slurry 18 total ionic activity and ionic activity for the nutrient being measured; or by utilizing the laser-induced breakdown spectroscopy method discussed above. Knowing the amount of water being added by the pump 14 also allows for soil salinity levels to be measured using sensors 34, such as a conductivity sensor.

The soil can be sampled from one location per analysis or multiple locations may be mixed together for analysis. Additionally, the present invention allows for soil to be sampled directly next to plants so that users can obtain data on soil conditions the plants are currently experiencing. The geographic location and depth of soil sample is recorded along with the soil analysis results by the computer 38. While the soil sample slurry 18 is preferably returned to the ground, it may be desirable to keep the soil sample slurry 18 for additional analysis or confirmation of any results. In this case, the soil sample slurry 18 may be stored in containers, such as a plastic cup and lid, a plastic bag or any suitable container that can be labeled with a sample identifier. The sample identifier may be a printed on bar code or an applied label containing identification information and potential location information.

A portable soil testing apparatus and method has thus been described. The present invention contemplates numerous variations, options, and alternatives, and is not to be limited to the specific embodiments described herein. For example, materials used to form the frame, tank, cup and the various components of the system may be varied. Sizes and volume capacities of the containers may be varied. In addition, the exact type of connections between the various components may also be varied according to size and availability and desirability. For example, pins, locks, screws, snaps, adhesives, buttons, hooks, or the like may be used to connect the various components to one the frame and wires of the appropriate size, shape, and material may be used. The exact form of connection shown and described is not to be limiting to the present invention. Other changes are considered to be part of the present invention.

What is claimed is:

1. A portable soil testing apparatus comprising:
a frame;
a water tank;
a water pump secured within the frame and operatively connected to the water tank;
a soil stirring station within the frame;
a sensor station within the frame;
a display secured within the frame;
a GPS antenna; and
a computer secured within the frame and operatively connected to the water pump, soil stirring station, sensor station, display and GPS antenna.

2. The portable soil testing apparatus of claim 1 wherein the sensor is an ion selective electrode.

3. The portable soil testing apparatus of claim 1 wherein the sensor is an optical sensor.

4. The portable soil testing apparatus of claim 1 wherein the sensor is a laser-induced breakdown spectroscopy sensor.

5. The portable soil testing apparatus of claim 1 further comprising a cup and a soil sample.

6. The portable soil testing apparatus of claim 1 wherein the frame is mounted to a vehicle.

7. The portable soil testing apparatus of claim 1 further comprising a calibrated amount of water dispensed by the pump.

8. The portable soil testing apparatus of claim 1 further comprising a battery operatively connected to the computer.

9. The portable soil testing apparatus of claim 1 wherein the display is a touch screen display.

10. A portable soil testing apparatus, the apparatus comprising:
a frame;
a soil sample;
a cup holding the soil sample;
a water tank secured within the frame;
a water pump connected to the water tank;
water dispensed from the water pump and into the cup;
a stirring rod;
a motor turning the stirring rod;
a slurry created by the motor turning the stirring rod and mixing the soil and the water;
a sensor;
an output signal from the sensor, the output signal created by operation of the sensor when the sensor is immersed in the slurry;
a computer to analyze the output signal;
a display operatively connected to the computer; and
a GPS antenna operatively connected to the computer.

11. The portable soil testing apparatus of claim 10 wherein the stirring rod and the sensor are located at a single station.

12. The portable soil testing apparatus of claim 10 wherein the stirring rod and the sensor are located at different stations.

13. The portable soil testing apparatus of claim 10 wherein the water dispensed from the water tank is a calibrated amount of water.

14. The portable soil testing apparatus of claim 10 further comprising a transceiver operatively connected to the computer.

15. The portable soil testing apparatus of claim 14 wherein the transceiver is a Bluetooth transceiver.

16. The portable soil testing apparatus of claim 10 wherein the computer includes a data storage medium.

17. A method of analyzing one or more soil properties, the method comprising:
taking a soil sample in a field;
placing the soil sample into a cup;
placing the cup under a nozzle;
operating a pump to transfer water from a tank and into the cup;
operating a motor to turn a stirring rod thereby stirring the water and the soil sample to create a slurry;
placing a sensor in the cup such that at least a portion of the sensor is immersed in the slurry;
operating the sensor;
detecting a property of the soil;
recording the soil property;
recording the location of the testing; and
displaying the results of the testing.

18. The method of claim 17 further comprising displaying instructions.

19. The method of claim 17 further comprising transmitting soil property and location to a user's personal digital device.

20. The method of claim 17 further comprising disposing of the slurry.

21. The method of claim 17 further comprising performing all of the steps of claim 17 while in a field.
